(19) United States
(12) Patent Application Publication
(10) Pub. No.: US 2021/0393521 A1
(43) Pub. Date: Dec. 23, 2021

(54) PREPARATION OF A PHARMACEUTICAL COMPOSITION OF OLODATEROL, TIOTROPIUM BROMIDE AND BUDESONIDE

(71) Applicants: Cai Gu Huang, Shrewsbury, MA (US); Jian Jiang Wu, Shanghai (CN)

(72) Inventors: Cai Gu Huang, Shrewsbury, MA (US); Jian Jiang Wu, Shanghai (CN)

(73) Assignee: Cai Gu Huang, Shrewsbury, MA (US)

(21) Appl. No.: 17/354,345

(22) Filed: Jun. 22, 2021

Related U.S. Application Data
(60) Provisional application No. 63/043,100, filed on Jun. 23, 2020.

(51) Int. Cl. A61K 9/08; A61K 31/5386; A61K 31/58; A61K 31/538; A61K 9/00; A61K 47/18; A61K 47/40; A61M 15/00; A61K 47/02; A61K 47/12

(52) U.S. Cl. CPC A61K 9/08; A61K 31/5386; A61K 31/58; A61K 31/538; A61K 47/12; A61K 47/186; A61K 47/40; A61M 15/0003; A61K 47/02; A61K 9/0078

(57) ABSTRACT

The present invention relates to a liquid pharmaceutical formulation and a method for administering a pharmaceutical formulation by nebulizing the pharmaceutical formulation in an inhaler. The propellant-free pharmaceutical preparation comprises: (a) budesonide, olodaterol and tiotropium bromide; (b) a solvent; (c) a pharmacologically acceptable solubilizing agent; (d) a pharmacologically acceptable preservative, (e) and a pharmacologically acceptable stabilizer, optionally including other pharmacologically acceptable additives.

### PRIORITY STATEMENT

This application claims the benefit of the filing date of U.S. Provisional Patent Application No. 63/043,100, filed on Jun. 23, 2020, which is incorporated herein by reference in its entirety.

### BACKGROUND OF THE INVENTION

Tiotropium bromide is chemically know as (1α,2β,4β,5α,7β-7-[(Hydroxydi-2-thienylacetyl)oxy]-9,9-dimethyl-3-oxa-9-azoniatricyclo[3.3.1.0(2,4)]nonane bromide monohydrate, and has the following chemical structure:

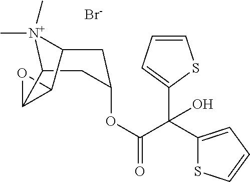 [SMILES: C[N+]1(C)C2CC(OC(=O)C(O)(c3cccs3)c3cccs3)CC1C1OC12 ; [Br-]]

Budesonide, also known as 11β,21-dihydroxy-16α,17α-(butylidenebis(oxy))pregna-1,4-diene-3,20-dione, is a synthetic pregnane steroid and non-halogenated cyclic ketal corticosteroid, having the following chemical structure:

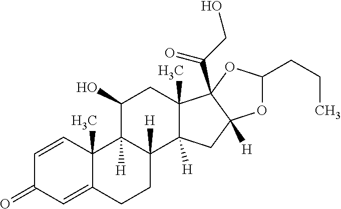

Olodaterol is chemically known as, 6-hydroxy-8-[(1R)-1-hydroxy-2-{[1-(4-methoxyphenyl)-2-methylpropan-2-yl]amino}ethyl]-3,4-dihydro-2H-1,4-benzoxazin-3-one, and has the following chemical structure:

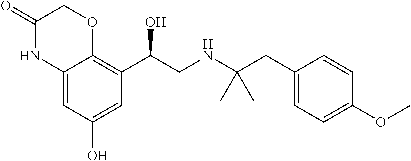 [SMILES: COc1ccc(CC(C)(C)NC[C@H](O)c2cc(O)cc3c2OCC(=O)N3)cc1]

Budesonide is a glucocorticoid with efficient local anti-inflammatory effect, it can strengthen the stability of endotheliocyte, smooth muscle cell and lysosome membrane, Immunosuppression reaction and the synthesis of reduction antibody, thus the release of the activity media such as histamine is reduced and active reduction, and can alleviate antigen-antibody in conjunction with time the enzymatic processes that excites, suppress the synthesis of bronchoconstriction material and release and alleviate the contractile response of smooth muscle.

Olodaterol is a novel, long-acting beta2-adrenergic agonist (LABA) that exerts its pharmacological effect by binding and activating beta2-adrenergic receptors located primarily in the lungs. Beta2-adrenergic receptors are membrane-bound receptors that are normally activated by endogenous epinephrine whose signaling, via a downstream L-type calcium channel interaction, mediates smooth muscle relaxation and bronchodilation. Activation of the receptor stimulates an associated G protein that then activates adenylate cyclase, catalyzing the formation of cyclic adenosine monophosphate (cAMP) and protein kinase A (PKA). Elevation of these two molecules induces bronchodilation by relaxation of airway smooth muscles. It is by this mechanism that olodaterol is used for the treatment of chronic obstructive pulmonary disease (COPD) and the progressive airflow obstruction that is characteristic of it. Treatment with bronchodilators helps to mitigate associated symptoms such as shortness of breath, cough, and sputum production.

Tiotropium is a long-acting, antimuscarinic bronchodilator used in the management of chronic obstructive pulmonary disease (COPD) and asthma. Tiotropium acts mainly on M3 muscarinic receptors located in the airways to produce smooth muscle relaxation and bronchodilation. Tiotropium bromide for inhalation is indicated for the maintenance of bronchospasm in COPD and to prevent exacerbations of COPD. A combination of tiotropium and olodaterol as a metered inhalation spray is indicated for maintenance of COPD. A tiotropium inhalation spray is indicated for the maintenance of bronchospasm in COPD, to prevent exacerbations of COPD, and to treat asthma in patients aged 12 or older. A tiotropium metered inhalation spray is indicated for the maintenance of bronchospasm in COPD, to prevent exacerbations of COPD, and to treat asthma in patients aged 6 or older.

These above compounds have valuable pharmacological properties. Budesonide, tiotropium bromide, and olodaterol can provide therapeutic benefit in the treatment of asthma or chronic obstructive pulmonary disease, including chronic bronchitis.

In one embodiment, the present invention relates to a propellant-free inhalable formulation comprising budesonide, tiotropium bromide and olodaterol dissolved in water, in combination with inactive ingredients that can be administered using a nebulization inhalation device, and the propellant-free inhalable aerosols resulting therefrom. The pharmaceutical formulations disclosed in the current invention are especially suitable for administration by nebulization inhalation, which has much better lung deposition (typically up to 55-60%, even up to 85-95%), compared to administration by a dry powder inhalation method.

The pharmaceutical formulation of the present invention is particularly suitable for administering the active substances by nebulization inhalation, especially for treating asthma and chronic obstructive pulmonary disease.

### SUMMARY OF THE INVENTION

The present invention relates to pharmaceutical formulations of budesonide, olodaterol, and tiotropium bromide, and pharmaceutically acceptable salts or solvates thereof, which can be administered by a nebulization inhalation method. The pharmaceutical formulations according to the invention meet high quality standards.

One aspect of the present invention is to provide an aqueous pharmaceutical formulation containing budesonide, olodaterol and tiotropium bromide, which meets the high standards needed in order to achieve optimum nebulization of the formulation using the inhalers mentioned hereinbefore. In one embodiment, the formulation is stable for at least about one year. In one embodiment, the formulation is stable for at least about two years. In one embodiment, the formulation is stable for at least about three years.

Another aspect is to provide propellant-free formulations, which can be solutions, containing budesonide, olodaterol, and tiotropium bromide which can be nebulized under pressure using an inhaler. In one embodiment, the inhaler is a nebulization inhaler device. The aerosol produced by the inhaler falls reproducibly within a specified range for particle size.

Another aspect of the invention is to provide pharmaceutical formulations, which can be solutions, comprising budesonide, olodaterol, and tiotropium bromide and other inactive excipients that can be administered by nebulization inhalation using an ultra-sonic based or air pressure based nebulizer/inhaler. In one embodiment, the formulation is stable for at least about 1 month. In one embodiment, the formulation is stable for at least about 6 months. In one embodiment, the formulation is stable for at least about one year. In one embodiment, the formulation is stable for at least about two years. In one embodiment, the formulation is stable for at least about three years.

More specifically, another aspect is to provide a stable pharmaceutical formulation of aqueous solutions containing budesonide, olodaterol, and tiotropium bromide and other excipients which can be administered using a nebulizer device. The formulations have substantially long term stability. In one embodiment, the formulations have a storage time of at least about 6 months at a temperature of from about 15° C. to about 25° C. In one embodiment, the formulations have a storage time of at least about 1 year at a temperature of from about 15° C. to about 25° C. In one embodiment, the formulations have a storage time of at least about 2 years at a temperature of from about 15° C. to about 25° C. In one embodiment, the formulations have a storage time of at least about 3 years at a temperature of from about 15° C. to about 25° C.

More specifically, another aspect of the current invention is to provide stable pharmaceutical formulations containing budesonide, olodaterol, and tiotropium bromide and other excipients which can be administered by nebulization inhalation using an ultrasonic jet or mesh nebulizer. In one embodiment, the formulations have a storage time of at least about 6 months at a temperature of from about 15° C. to about 25° C. In one embodiment, the formulations have a storage time of at least about 1 year at a temperature of from about 15° C. to about 25° C. In one embodiment, the formulations have a storage time of at least about 2 years at a temperature of from about 15° C. to about 25° C. In one embodiment, the formulations have a storage time of at least about 3 years at a temperature of from about 15° C. to about 25° C.

### BRIEF DESCRIPTION OF THE DRAWINGS

FIG. 1 depicts various nebulizers or components of a nebulizer. FIG. 1a depicts an ultrasonic nebulizer, FIG. 1b depicts a jet nebulizer, and FIG. 1c depicts the pressure vibration element and micropores of a mesh nebulizer.

### DETAILED DESCRIPTION OF THE INVENTION

Administering a liquid formulation without propellant gases using a suitable inhaler can achieve better delivery of the active substances to the lung. It is very important to increase lung deposition of a drug delivered by inhalation.

Currently, traditional pMDI or DPI (drying powder inhalation) only delivers about 20-30% of a drug into the lungs, resulting in a significant amount of drug being deposited on the month and throat, which can go into the stomach and cause unwanted side effects and/or secondary absorption through oral digest system.

Therefore, there is a need in the art to improve drug delivery by inhalation to significantly increase lung deposition. The soft mist or nebulization inhalation device disclosed in US20190030268 can significantly increase lung deposition of inhalable drugs.

Those inhalers can nebulize a small amount of a liquid formulation into an aerosol that is suitable for therapeutic inhalation within a few seconds. Those inhalers are particularly suitable for administering the liquid formulations disclosed herein.

In one embodiment, the nebulization devices useful for administering the aqueous pharmaceutical formulations of the present invention are those in which an amount of less than about 70 microliters of pharmaceutical solution can be nebulized in one puff so that the inhalable part of aerosol corresponds to the therapeutically effective quantity. In one embodiment, the nebulization devices useful for administering the aqueous pharmaceutical formulations of the present invention are those in which an amount of less than about 30 microliters of pharmaceutical solution can be nebulized in one puff so that the inhalable part of aerosol corresponds to the therapeutically effective quantity. In one embodiment, the nebulization devices useful for administering the aqueous pharmaceutical formulations of the present invention are those in which an amount of less than about 15 microliters of pharmaceutical solution can be nebulized in one puff so that the inhalable part of aerosol corresponds to the therapeutically effective quantity. In one embodiment, the average particle size of the aerosol formed from one puff is less than about 15 microns. In one embodiment, the average particle size of the aerosol formed from one puff is less than about 10 microns.

In one embodiment, the nebulization devices useful for administering the pharmaceutical formulations of the present invention are those in which an amount of less than about 8 milliliters of pharmaceutical solution can be nebulized in one puff so that the inhalable part of aerosol corresponds to the therapeutically effective quantity. In one embodiment, the nebulization devices useful for administering the pharmaceutical formulations of the present invention are those in which an amount of less than about 2 milliliters of pharmaceutical solution can be nebulized in one puff so that the inhalable part of aerosol corresponds to the therapeutically effective quantity. In one embodiment, the nebulization devices useful for administering the pharmaceutical formulations of the present invention are those in which an amount of less than about 1 milliliters of pharmaceutical solution can be nebulized in one puff so that the inhalable part of aerosol corresponds to the therapeutically effective quantity. In one embodiment, the average particle size of the aerosol formed from one puff is less than about 15 microns. In one embodiment, the average particle size of the aerosol formed from one puff is less than about 10 microns.

A device of this kind for the propellant-free administration of a metered amount of a liquid pharmaceutical formulation for inhalation is described in detail in, for example, US20190030268, entitled “inhalation atomizer comprising a blocking function and a counter”.

The pharmaceutical formulation contained in the nebulizer is converted into an aerosol destined for the lungs. The nebulizer uses high pressure to spray the pharmaceutical formulation.

The pharmaceutical solution is stored in a reservoir in this kind of inhaler. The formulations must not contain any ingredients that might interact with the inhaler to affect the pharmaceutical quality of the formulation or of the aerosol produced. In addition, the active substances in the pharmaceutical formulations are very stable when stored and can be administered directly.

In one embodiment, the formulations for use with the inhaler described above contains additives, such as the disodium salt of edetic acid (sodium edetate), to reduce the incidence of spray anomalies and to stabilize the formulation. In one embodiment, the formulations have a minimum concentration of sodium edetate.

One aspect of the present invention is to provide a pharmaceutical formulation containing budesonide, olodaterol, and tiotropium bromide, which meets the high standards needed to achieve optimum nebulization of the formulation using a nebulizer inhaler device. In one embodiment, the formulations have a storage time of at least about 6 months at a temperature of from about 15° C. to about 25° C. In one embodiment, the formulations have a storage time of at least about 1 year at a temperature of from about 15° C. to about 25° C. In one embodiment, the formulations have a storage time of at least about 2 years at a temperature of from about 15° C. to about 25° C. In one embodiment, the formulations have a storage time of at least about 3 years at a temperature of from about 15° C. to about 25° C.

Another aspect of the current invention is to provide propellant-free formulations containing budesonide, olodaterol, and tiotropium bromide, which are nebulized under pressure using an inhaler, wherein the particle size of the aerosol falls reproducibly within a specified range. In one embodiment, the inhaler is a nebulization inhaler. In one embodiment, the average particle size of the aerosol is less than about 10 μm.

Another aspect is to provide an aqueous pharmaceutical formulations containing budesonide, olodaterol, and tiotropium bromide and other inactive excipients which can be administered by inhalation.

According to the invention, any pharmaceutically acceptable salt or solvate of budesonide, olodaterol, and tiotropium may be used in the formulations. The terms budesonide, olodaterol, and tiotropium, as used herein, encompass budesonide, olodaterol, and tiotropium and pharmaceutically acceptable salts or solvates thereof. Although the specification frequently refers to tiotropium bromide, it is understood that salts of tiotropium, other than the bromide salt, can be used in the formulations. Tiotropium bromide is a preferred tiotropium salt.

In one embodiment, budesonide, olodaterol and tiotropium bromide are the active substances.

In one embodiment, the budesonide, olodaterol, and tiotropium bromide are dissolved in a solvent. In one embodiment, the solvent comprises water. In one embodiment, the solvent is water.

In one embodiment, a therapeutically effective dose of budesonide ranges from about 1 μg to about 640 μg. In one embodiment, a therapeutically effective dose of budesonide ranges from about 50 μg to about 640 μg. In one embodiment, a therapeutically effective dose of budesonide ranges from about 1 μg to about 100 μg. In one embodiment, a therapeutically effective dose of budesonide ranges from about 5 μg to about 50 μg. In one embodiment, a therapeutically effective dose of budesonide ranges from about 10 μg to about 30 μg. In one embodiment, a therapeutically effective dose of olodaterol ranges from about 3 μg to about 500 μg. In one embodiment, a therapeutically effective dose of olodaterol ranges from about 5 μg to about 500 μg. In one embodiment, a therapeutically effective dose of olodaterol ranges from about 10 μg to about 200 μg. In one embodiment, a therapeutically effective dose of olodaterol ranges from about 10 μg to about 80 μg. In one embodiment, a therapeutically effective dose of olodaterol ranges from about 3 μg to about 10 μg. In one embodiment, a therapeutically effective dose of tiotropium bromide ranges from about 1 μg to about 200 μg. In one embodiment, a therapeutically effective dose of tiotropium bromide ranges from about 1 μg to about 100 μg. In one embodiment, a therapeutically effective dose of tiotropium bromide ranges from about 1 μg to about 50 μg. In one embodiment, a therapeutically effective dose of tiotropium bromide ranges from about 5 μg to about 18 μg.

In one embodiment, the concentration of budesonide in the formulation ranges from about 1 mcg/ml to about 100 mcg/ml. In one embodiment, the concentration of budesonide in the formulation ranges from about 5 mcg/ml to about 100 mcg/ml. In one embodiment, the concentration of budesonide in the formulation ranges from about 10 mcg/ml to about 50 mcg/ml. In one embodiment, the concentration of olodaterol in the formulation ranges from about 2 mcg/ml to about 500 mcg/ml. In one embodiment, the concentration of olodaterol in the formulation ranges from about 10 mcg/ml to about 200 mcg/ml. In one embodiment, the concentration of olodaterol in the formulation ranges from about 30 mcg/ml to about 100 mcg/ml. In one embodiment, the concentration of tiotropium bromide in the formulation ranges from about 5 mcg/ml to about 150 mcg/ml. In one embodiment, the concentration of tiotropium bromide in the formulation ranges from about 5 mcg/ml to about 50 mcg/ml.

In one embodiment, the formulations include a pH adjusting agent, which can be an acid or a base. In one embodiment, the pH adjusting agent is selected from the group consisting of hydrochloric acid, citric acid, and salts thereof.

Other comparable pH adjusting agents can be used. In one embodiment, the pH adjusting agent is sodium hydroxide.

Selecting the proper pH of the formulation maximizes the stability of the active substances and/or other excipients. In one embodiment, the pH ranges from about 2.0 to about 6.0. In one embodiment, the pH ranges from about 3.0 to about 5.0. In one embodiment, the pH ranges from about 3.0 to about 4.0.

In one embodiment, the formulations according to the invention include a stabilizer or complexing agent. In one embodiment, the stabilizer or complexing agent is edetic acid (EDTA) or one of the known salts thereof, disodium edetate or edetate disodium dihydrate. In one embodiment the stabilizer or complexing agent is edetic acid and/or a salt thereof.

Other comparable stabilizers or complexing agents can be used. Other suitable stabilizers or complexing agents include, but are not limited to citric acid, edetate disodium, and edetate disodium dihydrate.

The terms “stabilizer” and “complexing agent,” as used herein, mean a molecule which is capable of entering into complex bonds. Preferably, these compounds have the effect of complexing cations. In one embodiment, the concentration of the stabilizer or complexing agent ranges from about 0.04 mg/4 ml to about 20 mg/4 ml. In one embodiment, the concentration of the stabilizer or complexing agent ranges from about 0.2 mg/4 ml to about 8 mg/4 ml. In one embodiment, the stabilizer or complexing agent is edetate disodium dihydrate in an amount of about 0.4 mg/4 ml.

In one embodiment, all of the ingredients of the formulation are present in solution.

The term “additives,” as used herein, means any pharmacologically acceptable and therapeutically useful substance which is not an active substance, but can be formulated together with the active substances in the pharmacologically suitable solvent, in order to improve the qualities of the formulation. Preferably, these substances have no pharmacological effects or no appreciable, or at least no undesirable, pharmacological effects, in the context of the desired therapy.

Suitable additives include, but are not limited to, other stabilizers, complexing agents, antioxidants, surfactants, and/or preservatives which prolong the shelf life of the finished pharmaceutical formulation, vitamins, and other additives known in the art.

Suitable preservatives can be added to protect the formulation from contamination with pathogenic bacteria. Suitable preservatives include, but are not limited to, benzalkonium chloride, benzoic acid, and sodium benzoate. In one embodiment, the formulations contain only benzalkonium chloride. In one embodiment, the preservative is present in an amount ranging from about 0.08 mg/4 ml to about 12 mg/4 ml. In one embodiment, the preservative is benzalkonium chloride in an amount of about 0.4 mg/4 ml.

In one embodiment, the formulations include a solubility enhancing agent. In one embodiment, the solubility enhancing agent is selected from Tween 80 and cyclodextrin derivatives. In one embodiment, the solubility enhancing agent is a cyclodextrin derivative or a known salt thereof. The solubility enhancing agent aids the solubility of the active ingredients or other excipients. In one embodiment, the solubility enhancing agent is sulfobutylether β-cyclodextrin or a salt thereof. In one embodiment, the solubility enhancing agent is present in an amount ranging from about 1 g/100 ml to about 40 g/100 ml.

In one embodiment, the formulations for administration by nebulization include a surfactant or other solubility enhancing agents. In one embodiment, the surfactant or solubility enhancing agent is selected from the group consisting of Tween 80 and cyclodextrin derivatives. In one embodiment, the surfactant or solubility enhancing agent is a cyclodextrin derivative or a known salt thereof. In one embodiment, the surfactant or solubility enhancing agent is sulfobutylether β-cyclodextrin. In one embodiment, the sulfobutylether β-cyclodextrin is present in an amount ranging from about 0.04 g/4 ml to about 1.6 g/4 ml. In one embodiment, the surfactant or solubility enhancing agent is sulfobutylether β-cyclodextrin in an amount of about 0.8 g/4 ml.

Another aspect of the current invention is to provide stable pharmaceutical formulations containing budesonide, olodaterol, and tiotropium bromide and other excipients that can be administered by nebulization using an inhaler. In one embodiment, the formulations have substantially long term stability. In one embodiment, the formulations have a storage time of at least about 6 months at a temperature of from about 15° C. to about 25° C. In one embodiment, the formulations have a storage time of at least about 1 year at a temperature of from about 15° C. to about 25° C. In one embodiment, the formulations have a storage time of at least about 2 years at a temperature of from about 15° C. to about 25° C. In one embodiment, the formulations have a storage time of at least about 3 years at a temperature of from about 15° C. to about 25° C.

Another aspect of the current invention is to provide pharmaceutical formulations, which can be solutions, comprising budesonide, olodaterol, and tiotropium bromide and other inactive excipients which can be administered by nebulization inhalation using an ultra-sonic based or air pressure based nebulizer/inhaler. In one embodiment, the formulations have a storage time of few months. In one embodiment, the formulations have a storage time of about 1 to about 6 months. In one embodiment, the formulations have a storage time of about one year. In one embodiment, the formulations have a storage time of about two years. In one embodiment, the formulations have a storage time of about three years.

More specifically, another aspect of the current invention is to provide stable pharmaceutical formulations containing budesonide, olodaterol, and tiotropium bromide and other excipients which can be administered by nebulization inhalation using an ultra-sonic based or air pressure based nebulizers/inhalers. In one embodiment, the formulations have substantially long term stability. In one embodiment, the formulations have a storage time of at least about 6 months at a temperature of from about 15° C. to about 25° C. In one embodiment, the formulations have a storage time of at least about 1 year at a temperature of from about 15° C. to about 25° C. In one embodiment, the formulations have a storage time of at least about 2 years at a temperature of from about 15° C. to about 25° C. In one embodiment, the formulations have a storage time of at least about 3 years at a temperature of from about 15° C. to about 25° C.

In one embodiment, the formulations include sodium chloride. In one embodiment, the concentration the sodium chloride ranges from about 0.1 g/100 ml to about 0.9 g/100 ml.

In one embodiment, the concentration of budesonide in the formulation ranges from about 1 mcg/ml to about 100 mcg/ml. In one embodiment, the concentration of budesonide in the formulation ranges from about 5 mcg/ml to about 100 mcg/ml. In one embodiment, the concentration of budesonide in the formulation ranges from about 10 mcg/ml to about 50 mcg/ml. In one embodiment, the concentration of olodaterol in the formulation ranges from about 2 mcg/ml to about 500 mcg/ml. In one embodiment, the concentration of olodaterol in the formulation ranges from about 10 mcg/ml to about 200 mcg/ml. In one embodiment, the concentration of olodaterol in the formulation ranges from about 30 mcg/ml to about 100 mcg/ml. In one embodiment, the concentration of tiotropium bromide in the formulation ranges from about 5 mcg/ml to about 150 mcg/ml. In one embodiment, the concentration of tiotropium bromide in the formulation ranges from about 5mcg/m1 to about 50 mcg/ml.

In one embodiment, the formulations include a surfactant or other solubility enhancing agent. In one embodiment, the surfactant or solubility enhancing agent is selected from the group consisting of Tween 80 and cyclodextrin derivatives. In one embodiment, the surfactant or solubility enhancing agent is a cyclodextrin derivatives or a known salt thereof. In one embodiment, the surfactant or solubility enhancing agent is sulfobutylether β-cyclodextrin.

In one embodiment, the formulations include a surfactant or other solubility enhancing agent. In one embodiment, the surfactant or solubility enhancing agent is selected from the group consisting of Tween 80 and cyclodextrin derivative. In one embodiment, the surfactant or solubility enhancing agent is a cyclodextrin derivatives or a known salt thereof. In one embodiment, the surfactant or solubility enhancing agent is sulfobutylether β-cyclodextrin. In one embodiment, the surfactant or solubility enhancing agent is sulfobutylether β-cyclodextrin in an amount ranging from about 5 mg/ml to about 0.4 g/ml. In one embodiment, the surfactant or solubility enhancing agent is sulfobutylether β-cyclodextrin in a concentration of about 0.2 g/ml.

It has been found that, advantageously, sulfobutylether β-cyclodextrin not only has the effect of enhancing solubility, but has the effect of improving the stability of the active ingredients.

Another aspect of the current invention is to provide stable pharmaceutical formulations wherein the concentration of tiotropium bromide in the formulation ranges from about 5 mcg/ml to about 150 mcg/ml which can be administered with a nebulizer. Another aspect of the current invention is to provide stable pharmaceutical formulations wherein the concentration of tiotropium bromide in the formulation ranges from about 5 mcg/ml to about 50 mcg/ml which can be administered with a nebulizer. In one embodiment, the concentration of budesonide in the formulation ranges from about 1 mcg/ml to about 100 mcg/ml which can be administered with a nebulizer. In one embodiment, the concentration of budesonide in the formulation ranges from about 5 mcg/ml to about 100 mcg/ml which can be administered with a nebulizer. In one embodiment, the concentration of budesonide in the formulation ranges from about 10 mcg/ml to about 50 mcg/ml which can be administered with a nebulizer. In one embodiment, the concentration of olodaterol in the formulation ranges from about 2 mcg/ml to about 500 mcg/ml which can be administered with a nebulizer. In one embodiment, the concentration of olodaterol in the formulation ranges from about 10 mcg/ml to about 200 mcg/ml which can be administered with a nebulizer. In one embodiment, the concentration of olodaterol in the formulation ranges from about 30 mcg/ml to about 100 mcg/ml which can be administered with a nebulizer. In one embodiment, the formulations have substantially long-term stability. In one embodiment, the formulations have a storage time of at least about 6 months at a temperature of from about 15° C. to about 25° C. In one embodiment, the formulations have a storage time of at least about 1 year at a temperature of from about 15° C. to about 25° C. In one embodiment, the formulations have a storage time of at least about 2 years at a temperature of from about 15° C. to about 25° C. In one embodiment, the formulations have a storage time of at least about 3 years at a temperature of from about 15° C. to about 25° C.

In one embodiment, the pH value of the formulations for nebulization range from about 3 to about 6. In one embodiment, the pH value of the formulations for nebulization range from about 3 to about 5. In one embodiment, the pH value of the formulations for nebulization range from about 3 to about 4.

In one embodiment, the formulations according to the invention are filled into canisters to form a highly stable formulation for use in a nebulization device. The formulations exhibit substantially no particle growth, change of morphology, or precipitation. There is also no, or substantially no, problem of deposition of suspended particles on the surface of the canisters or the valves, so that the formulations can be discharged from a suitable nebulization device with high dose uniformity. The nebulizer can be selected from an ultrasonic nebulizer; a jet nebulizer; or a mesh nebulizer, such as Pari eFlow nebulization inhaler, or other commercially available ultrasonic nebulizer, jet nebulizer or mesh nebulizer.

The pharmaceutical formulation is converted by the nebulizer into an aerosol destined for the lungs. The nebulizer uses high pressure to the spray the pharmaceutical formulation.

Nebulizers are instruments that generate very fine particles of a liquid in a gas. As is well known, particles intended for treatment of the lower airway, i.e., the bronchial tree or the lungs, are generally less than 10 micrometers in the largest dimension, to prevent unwanted deposition onto surfaces of the mouth and pharynx, and more preferably are less than 5 μm in the largest dimension, in order to achieve the desired pharmacological effect. Particles much smaller than about 0.5 μm in the largest dimension frequently are not easily deposited at the desired location, and a large fraction of these particles will simply be exhaled by the patient. For these reasons, it is generally desired to produce particles which average between about 1 μm and about 5 μm in their largest dimension, while minimizing production of particles having sizes less than about 0.5 μm or greater than about 10 μm. A preferred average particle size ranges from about 0.5 μm to about 5 μm.

Nebulization, although used more infrequently than other drug delivery techniques, has certain advantages for special patient groups, such as young children and the very infirm. Although somewhat cumbersome equipment is needed and there may be more stringent cleaning requirements than exist for other delivery techniques, no particular patient skill or coordination is required; the patient merely needs to breathe normally to introduce the medication into the airway. Thus, treatment can be delivered even to an unconscious patient or an infant. Another advantage of administering drugs using a nebulizer is that quantities of moisture are delivered to the airway; which can help to fluidize secretions and increase patient comfort.

In one embodiment, the pharmaceutical formulations of the present invention are administered using a nebulizer wherein less than about 8 milliliters of pharmaceutical solution can be nebulized in one puff so that the inhalable part of aerosol corresponds to the therapeutically effective quantity. In one embodiment, the pharmaceutical formulations of the present invention are administered using a nebulizer wherein less than about 2 milliliters of pharmaceutical solution can be nebulized in one puff so that the inhalable part of aerosol corresponds to the therapeutically effective quantity. In one embodiment, the pharmaceutical formulations of the present invention are administered using a nebulizer wherein less than about 1 milliliters of pharmaceutical solution can be nebulized in one puff so that the inhalable part of aerosol corresponds to the therapeutically effective quantity. In one embodiment, the average particle size of the aerosol formed from one puff is less than about 15 microns. In one embodiment, the average particle size of the aerosol formed from one puff is less than about 10 microns.

A device of this kind for the propellant-free administration of a metered amount of a liquid pharmaceutical composition for inhalation is described in detail in, for example, US20190030268, entitled “inhalation atomizer comprising a blocking function and a counter”.

The pharmaceutical formulation is converted by the nebulizer into aerosol destined for the lungs. The nebulizer uses high pressure to spray the pharmaceutical formulation.

The pharmaceutical formulation is stored in a reservoir in this kind of inhaler. The formulations must not contain any ingredients which might interact with the inhaler to affect the pharmaceutical quality of the solution or of the aerosol produced. In one embodiment, the active substances in pharmaceutical formulations are very stable when stored and can be administered directly.

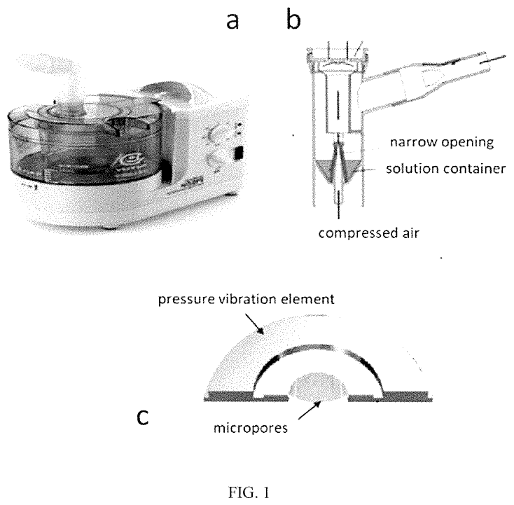

An ultrasonic energy can atomize a water-soluble drug into tiny mist particles having a particle size ranging from about 1 um to about 5 um at normal temperature. A jet nebulizer includes a compressed air source and an atomizer. The compressed air is suddenly decompressed after passing through the narrow opening at high speed, a negative pressure is generated locally, and the drug solution is sucked out of the container because of a siphon effect. When subjected to high-speed air flow, the drug solution is broken into small aerosol particles by collision. Mesh nebulizer contains a stainless steel mesh covered with micropores having a diameter of about 3 μm. The number of micropores on the stainless steel mesh, which is conical with the cone bottom facing the liquid surface, exceeds 1000. FIG. 1a depicts an ultrasonic nebulizer. FIG. 1b depicts a jet nebulizer. FIG. 1c depicts the pressure vibration element and micropores of a mesh nebulizer.

Olodaterol is selective fast-acting β 2-adrenergic receptor agonist, chemically known as 6-hydroxy-8-[(1R)-1-hydroxy-2-{[1-(4-methoxyphenyl)-2-methylpropan-2-yl] amino}ethyl]-3,4-dihydro-2H-1,4-benzoxazin-3-one, that exhibits high selectivity to the β 2-adrenergic receptor (abbreviated beta 2-receptor), exhibits rapid onset of action, has a long half-life (more than 12 h), and can maintain bronchiectatic activity for 24 h.

Budesonide is a glucocorticoid with efficient local anti-inflammatory effect, it can strengthen the stability of endotheliocyte, smooth muscle cell and lysosome membrane, Immunosuppression reaction and the synthesis of reduction antibody, thus the release of the activity media such as histamine is reduced and active reduction, and can alleviate antigen-antibody in conjunction with time the enzymatic processes that excites, suppress the synthesis of bronchoconstriction material and release and alleviate the contractile response of smooth muscle.

Tiotropium is a long-acting, antimuscarinic bronchodilator used in the management of chronic obstructive pulmonary disease (COPD) and asthma. Tiotropium acts mainly on M3 muscarinic receptors located in the airways to produce smooth muscle relaxation and bronchodilation. Tiotropium bromide for inhalation is indicated for the maintenance of bronchospasm in COPD and to prevent exacerbations of COPD. A combination of tiotropium and olodaterol as a metered inhalation spray is indicated for maintenance of COPD. A tiotropium inhalation spray is indicated for the maintenance of bronchospasm in COPD, to prevent exacerbations of COPD, and to treat asthma in patients aged 12 or more years old. A tiotropium metered inhalation spray is indicated for the maintenance of bronchospasm in COPD, to prevent exacerbations of COPD, and to treat asthma in patients aged 6 or more years old.

### EXAMPLES

Materials and reagents:

50% benzalkonium chloride aqueous solution was purchased from Merck.

Edetate disodium dihydrate was purchased from Merck.

Sodium hydroxide was purchased from Titan reagents.

Hydrochloric acid was purchased from Titan reagents.

Citric acid purchased was from Merck.

Sodium chloride purchased was from Merck.

Sulfobutylether β-cyclodextrin was purchased from Zhiyuan Bio-tech Co., Ltd., China.

### Example 1

Solubility test to investigate the solubilizing effect of SBECD and Tween 80 as solubilizers on BD, and to investigate the solubility of BD at different concentrations.

Investigation of the solubility of BD under different concentrations of SBECD: Weigh 0.1 g, 0.3 g, 0.5 g, and 1.0 g of BD into a 10 ml EP tube, add 10 ml of pure water, and shake until the BD is completely dissolved, then add excess BD (about 500 mg/100 mL), and wrap the EP tube in tin foil to protect from light. After protecting from light, place the EP tube on a shaker, shake for 24 hours, and centrifuge to collect the supernatant.

Investigation of the solubility of BD under different concentrations of Tween 80: Weigh 0.002 g, 0.001 g, 0.0005 g, 0.1 g, 0.3 g, 0.5 g, and 1.0 g of BD into a weighing bottle, transfer the BD to a 10 ml EP tube by rinsing the beaker with sufficient water to provide 10 mL in the EP tube, add excess BD (about 500 mg/100 mL), and wrap the EP tube in tin foil to protect it from light, place the EP tube on a shaker and shake for 24 hours, then centrifuge to collect the supernatant.

TABLE 1
Solubility of BD in Different Concentrations of SBECD and
Different Concentrations of Tween 80

| SBECD | BD Solubility | Tween 80 | BD Solubility |
|---|---|---|---|
| concentration | (mg/100 ml) | concentration | (mg/100 ml) |
| 1% | 16.91 | 1% | 16.54 |
| 3% | 40.28 | 3% | 38.88 |
| 5% | 61.91 | 5% | 64.84 |
| 10% | 81.3 | 10% | 118.79 |
| | | 5 mg/100 ml | 0.224 |
| | | 10 mg/100 ml | 0.256 |
| | | 20 mg/100 ml | 0.292 |

According to the above results, it can be seen that SBECD and Tween 80 have a similar solubilization effect on BD. Tween 80 is within acceptable limits. According to the US pharmacopoeia, the concentration of Tween 80 should not exceed 20 mg/100 ml in an inhalation suspension. The solubility of BD in Tween 80 concentrations of 20 mg/100 ml is only 2.92 μg/ml. Unable to meet the requirements. A BD solubility of about 500 μg/ml is needed.

### Example 2

pH Stability:

TABLE 2
Ingredient of Samples 1-5

| Ingredients | Sample 1 | | Sample 2 | | Sample 3 | | Sample 4 | | Sample 5 | |
|---|---|---|---|---|---|---|---|---|---|---|
| Tiotropium | 2.063 | mg | 2.063 | mg | 2.063 | mg | 2.063 | mg | 2.063 | mg |
| bromide(TB) | | | | | | | | | | |
| Olodaterol | 1.842 | mg | 1.842 | mg | 1.842 | mg | 1.842 | mg | 1.842 | mg |
| hydrochloride | | | | | | | | | | |
| (OH) | | | | | | | | | | |
| Budesonide | 53.33 | mg | 53.33 | mg | 53.33 | mg | 53.33 | mg | 53.33 | mg |
| (BD) | | | | | | | | | | |
| Sulfo- | 10.00 | g | 10.00 | g | 10.00 | g | 10.00 | g | 10.00 | g |
| butylether | | | | | | | | | | |
| β- | | | | | | | | | | |
| Cyclodextrin | | | | | | | | | | |
| sodium | | | | | | | | | | |
| (SBECD) | | | | | | | | | | |
| HCl | Adjusted | | Adjusted | | Adjusted | | Adjusted | | Adjusted | |
| | to pH 5.5 | | to pH 5.0 | | to pH 4.5 | | to pH 4.0 | | to pH 3.5 | |
| Purified | Added to | | Added to | | Added to | | Added to | | Added to | |
| water | 104.7 | g | 104.7 | g | 104.7 | g | 104.7 | g | 104.7 | g |

The formulation and preparation of samples 1-5 for administration by nebulization inhalation is as follows:
1: Weigh the prescribed amount of SBECD according to Table 2 into an empty beaker, add 93 g of purified water and dissolve, and, after dissolving, adjust the pH according to Table 2 with hydrochloric acid
2: Weigh the prescribed amount of BD according to Table 2 into the above solution and stir in the dark to dissolve
3: After dissolving, add TB and OH according to the amounts in Table 2, adjust the pH to 5.5 with hydrochloric acid, and add water to 104.7 g
4: Adjust the pH to the target pH according to Table 2 with hydrochloric acid and designate the samples as Sample_1, Sample_2, Sample_3, Sample_4, and Sample_5.
5: Divide each sample into parts and store at 40° C. or 60° C. for 5 days and 10 days.
At each time point detect impurities.

Impurities Test Method:
Mobile phase A: accurately weigh 3.17 g of sodium dihydrogen phosphate, add 1L of pure water to dissolve, adjust the pH of the resulting solution to 3.20 with phosphoric acid.
Mobile Phase B: Acetonitrile
Instrument ID: HPLC waters_M (2695/2996) S/N: K06SM4612R,
Column ID: YMC-Triart C18 250*4.6 5 um S/N: 117DA90183
Detection wavelength: 230 nm
flow rate: 1.0 mL/min
column temperature: 40° C.
Injection volume: 100 μl
Running time: 35 min.
Gradient elution:

| Time | Mobile phase A % | Mobile Phase B % |
|---|---|---|
| 0 | 70 | 30 |
| 7 | 70 | 30 |
| 8 | 60 | 40 |
| 32 | 60 | 40 |
| 33 | 70 | 30 |
| 35 | 70 | 30 |

The test results are shown below.

TABLE 3
Thermal Stability at 60° C. of sample 1-5 (Condition: 60° C. ± 2° C./RH 75%)

| | | | Sample 1 | Sample 2 | Sample 3 | Sample 4 | Sample 5 |
|---|---|---|---|---|---|---|---|
| Ingredients | | | pH 5.5 | pH 5 | pH 4.5 | pH 4.0 | pH 3.5 |
| 0 day | Character | | Colorless clear liquid | | | | |
| | Content | OH | 16.92 | 17.15 | 17.14 | 17.11 | 16.66 |
| | μg/ml | BD | 493.4 | 500.2 | 500.1 | 499.4 | 495.9 |
| | | TB | 20.69 | 20.97 | 20.95 | 20.96 | 20.78 |
| | Total | OH | ND | ND | ND | ND | ND |
| | impurities % | BD | 0.55 | 0.55 | 0.55 | 0.54 | 0.55 |
| | | TB | ND | ND | ND | ND | ND |
| 60° C. | Character | | Colorless clear liquid | | | | |
| 5 days | Content | OH | 16.34 | 16.31 | 16.06 | 15.29 | 14.17 |
| | μg/ml | BD | 487.6 | 491.5 | 491.6 | 490.3 | 487.7 |
| | | TB | 20.31 | 20.71 | 20.7 | 20.81 | 21.02 |
| | Total | OH | 0.59 | 0.55 | 0.58 | 0.78 | 1.27 |
| | impurities % | BD | 1.45 | 1.46 | 1.36 | 1.51 | 1.54 |
| | | TB | 0.8 | 0.57 | 0.23 | 0.1 | 0.12 |
| 60° C. | Character | | Colorless clear liquid | | | | |
| 10 days | Content | OH | 16.34 | 16.31 | 16.06 | 15.29 | 14.17 |
| | μg/ml | BD | 487.6 | 491.5 | 491.6 | 490.3 | 487.7 |
| | | TB | 20.31 | 20.71 | 20.7 | 20.81 | 21.02 |
| | Total | OH | 3.84 | 3.8 | 3.65 | 8.73 | 11.54 |
| | impurities % | BD | 2.32 | 1.59 | 2.15 | 2.97 | 2.85 |
| | | TB | 2.19 | 1.7 | 0.95 | 0.35 | 0.41 |

TABLE 4
Thermal Stability at 40° C. of sample 1-5 (Condition: 40° C. ± 2° C./RH 75%)

| | | | Sample 1 | Sample2 | Sample 3 | Sample 4 | Sample 5 |
|---|---|---|---|---|---|---|---|
| Ingredients | | | pH 5.5 | pH 5.0 | pH 4.5 | pH 4.0 | pH 3.5 |
| 0 day | Character | | Colorless clear liquid | | | | |
| | Content | OH | 16.92 | 17.15 | 17.14 | 17.11 | 16.66 |
| | μg/ml | BD | 493.4 | 500.2 | 500.1 | 499.4 | 495.9 |
| | | TB | 20.69 | 20.43 | 20.95 | 20.96 | 20.78 |
| | Total | TB | ND | ND | ND | ND | ND |
| | impurities % | OH | ND | ND | ND | ND | ND |
| | | BD | 0.55 | 0.55 | 0.03 | 0.54 | 0.55 |
| 40° C. | Character | | Colorless clear liquid | | | | |
| 5 days | Content | OH | 17.03 | 16.96 | 16.92 | 16.64 | 15.73 |
| | μg/ml | BD | 496.5 | 496.6 | 497.6 | 496.5 | 494.2 |
| | | TB | 20.43 | 20.47 | 20.54 | 20.6 | 20.78 |
| | Total | TB | 0.28 | 0.08 | ND | ND | ND |
| | impurities % | OH | ND | ND | 0.05 | 0.1 | 0.25 |
| | | BD | 0.85 | 0.82 | 0.76 | 0.76 | 0.81 |
| 40° C. | Character | | Colorless clear liquid | | | | |
| 10 | Total | TB | 0.5 | 3.17 | 0.16 | ND | ND |
| days | impurities % | OH | 0.57 | 0.56 | 0.85 | 1.59 | 3.1 |
| | | BD | 1.01 | 1.71 | 0.88 | 0.92 | 0.99 |

Hydrochloric acid was used to adjust the pH. The total impurities are as shown in the above table. At different pH values, each active ingredient has a different degree of degradation. The formulation at pH 4.5 was the most stable.

TABLE 5
Ingredient of Sample 6-9

| Ingredients | Sample 6 | | Sample 7 | | Sample 8 | | Sample 9 | |
|---|---|---|---|---|---|---|---|---|
| Tiotropium | 2.063 | mg | 2.063 | mg | 2.063 | mg | 2.063 | mg |
| bromide (TB) | | | | | | | | |
| Olodaterol (OH) | 1.842 | mg | 1.842 | mg | 1.842 | mg | 1.842 | mg |
| Budesonide (BD) | 53.33 | mg | 53.33 | mg | 53.33 | mg | 53.33 | mg |
| Sulfobutylether | 10.00 | g | 10.00 | g | 10.00 | g | 10.00 | g |
| (β-Cyclodextrin | | | | | | | | |
| (SBECD) | | | | | | | | |
| Citric acid (CA) | Adjusted | | Adjusted | | Adjusted | | Adjusted | |
| | to pH 4.0 | | to pH 3.5 | | to pH3.0 | | to pH 2.5 | |
| Purified water | Added to | | Added to | | Added to | | Added to | |
| | 104.7 | g | 104.7 | g | 104.7 | g | 104.7 | g |

The formulation and preparation of samples 6-9 for administration by nebulization inhalation is as follows:
1. Weigh the prescribed amount of SBECD into a beaker and add sufficient water to make the weight up to 104.70 g,
2. After stirring to dissolve the SBECD, adjust the pH to about 4.00 with CA.
3. After adjusting the pH, add the prescribed amount of BD, and stir overnight in the dark to dissolve.
4. On the following day, after the BD is dissolved, add the prescribed amount of TB and OH and filter after dissolving.
5. Adjust the pH to 4.0, 3.5, 3.0, and 2.5 with CA. Designate the samples as Sample 6, Sample 7, Sample 8, and Sample 9, respectively.
6. Divide each sample into parts and place them at 60° C.
Determine impurities as days 0, 7, and 14.

TABLE 6
Thermal Stability at 60° C. of Samples 6-9 (Condition: 60° C. ± 2° C./RH 75%)

| | | | Sample 6 | Sample 7 | Sample 8 | Sample 9 |
|---|---|---|---|---|---|---|
| Ingredients | | | pH 4.0 | pH 3.5 | pH 3.0 | pH 2.5 |
| 0 day | Character | | Colorless clear liquid | | | |
| | Content | TB | 20.68 | 21.01 | 20.99 | 21.38 |
| | μg/ml | OH | 18.11 | 18.27 | 18.16 | 18.52 |
| | | BD | 534.74 | 543.01 | 542.53 | 553.37 |
| | Total | TB | NA | NA | NA | NA |
| | impurities % | OH | 0.08 | 0.06 | 0.06 | 0.08 |
| | | BD | NA | NA | NA | NA |
| 60° C. | Character | | Colorless clear liquid | | | |
| 7 days | Content | TB | 20.33 | 20.45 | 20.17 | 20.2 |
| | μg/ml | OH | 17.9 | 17.73 | 15.97 | 14.97 |
| | | BD | 522.05 | 519.22 | 514.49 | 499.91 |
| | Total | TB | 0.21 | NA | NA | NA |
| | impurities % | OH | 0.95 | 1.8 | 7.59 | 8.89 |
| | | BD | 0.76 | 1.01 | 1.58 | 3.26 |
| 60° C. | Character | | Colorless clear liquid | | | |
| 14 days | Content | TB | 19.09 | 18.68 | 18.81 | 18.84 |
| | μg/ml | OH | 17.09 | 17.3 | 17.64 | 16.48 |
| | | BD | 523.32 | 532.95 | 543.68 | 487.37 |
| | | BD | 0.68 | 0.75 | 0.81 | 1.14 |
| | Total | TB | 0.43 | NA | NA | 0.78 |
| | impurities % | OH | 1.57 | 1.69 | 8 | 10.65 |
| | | BD | 1.73 | 1.49 | 3.47 | 6.32 |

Citric acid was used to adjust the pH. The total impurities are shown in Table 6. At different pH values, each active ingredient has a different degree of degradation. pH 4.0 exhibits the best stability.

Comparative analysis of results: When citric acid and hydrochloric acid are used to adjust the pH, the best stability is observed at pH 4.0 and pH 4.5, respectively. When the two acids are compared, the best stability is exhibited at pH 4.0 with citric acid.

### Example 3

Aerodynamic Particle Size Distribution:

The aerodynamic particle size distribution of Sample 3 of Example 1 was determined using a Next Generation Pharmaceutical Impactor (NGI).

The device used to aerosolize the formulation was a PARI E-flow, purchased from PARI. The device was held close to the NGI inlet until no aerosol was visible. The flow rate of the NGI was set to 15 L/minute and was operated under ambient temperature and a relative humidity (RH) of 90%.

The solution of sample 3 was discharged into the NGI. Fractions of the dose were deposited at different stages of the NGI, in accordance with the particle size of the fraction. Each fraction was washed from the stage and analyzed using HPLC.

TABLE 7
Aerodynamic Particle Size Distribution of Sample 3

| | OH | | BD | | TB | | |
|---|---|---|---|---|---|---|---|
| | | Percentage | | Percentage | | Percentage | Cut-off |
| | | content | | content | | content | diameters |
| | | at all | | at all | | at all | at 15 |
| | Dosage | levels | Dosage | levels | Dosage | levels | L/min |
| | (μg) | % | (μg) | % | (μg) | % | (μm) |
| Device | 1 | 20.45 | 28.61 | 19.20 | 0.95 | 19.27 | |
| Throat | 0 | 0 | 0 | 0.00 | 0 | 0 | |
| Stage 1 | 0.11 | 2.25 | 3.73 | 2.50 | 0.14 | 2.84 | 14.10 |
| Stage 2 | 0.14 | 2.86 | 4.62 | 3.10 | 0.16 | 3.25 | 8.61 |
| Stage 3 | 0.72 | 14.72 | 21.43 | 14.38 | 0.72 | 14.60 | 5.39 |
| Stage 4 | 1.53 | 31.29 | 45.41 | 30.47 | 1.49 | 30.22 | 3.30 |
| Stage 5 | 0.94 | 19.22 | 28.06 | 18.83 | 0.89 | 18.05 | 2.08 |
| Stage 6 | 0.33 | 6.75 | 10.06 | 6.75 | 0.31 | 6.29 | 1.36 |
| Stage 7 | 0.12 | 2.45 | 3.72 | 2.50 | 0.11 | 2.23 | 0.98 |
| MOC | 0 | 0 | 0.51 | 0.34 | 0 | 0 | |
| Filter | 0 | 0 | 2.87 | 1.93 | 0.16 | 3.25 | |

| | | | |
|---|---|---|---|
| MMAD | 3.92 | 3.86 | 3.89 |
| (μm) | | | |
| GSD | 1.6 | 1.63 | 1.64 |
| Theoretical | 5.52 | 160.53 | 6.12 |
| dose (μg) | | | |
| Actual test | 4.89 | 149.02 | 4.93 |
| dose (μg) | | | |
| Recovery | 88.59 | 92.83 | 80.56 |
| rate (%) | | | |
| ISM (μg) | 3.78 | 116.68 | 3.84 |
| FPD (μg) | 2.92 | 90.63 | 2.96 |
| FPF (%) | 59.71 | 60.82 | 60.04 |

MOC is Micro-Orifice Collector.
ISM is Impactor Size Mass.
FPF is Fine Particle Fraction.
FPD is Fine Particle dose.
MMAD is mass median aerodynamic diameter.
GSD is Geometric Standard Deviation.
Stage Filter, which is a DDU tube connected to the end of the NGI.

### Comparative Example 1

Aerodynamic Particle Size Distribution of a budesonide suspension (Comparative Sample 1 (Pulmicort): batch number: LOT 324439;dosage: 0.5 mg; Specification: 2 ml/inhalation/time).

The budesonide suspension sample was purchased from AstraZeneca Pty Ltd.

The aerodynamic particle size distribution was determined using a Next Generation Pharmaceutical Impactor (NGI). The Sample is Comparative Sample 1 (Pulmicort). The device used to aerosolize the formulation was an LC-Plus, purchased from PARI in Germany. The device was held close to the NGI inlet until no aerosol was visible. The flow rate of the NGI was set to 30 L/minute and was operated under ambient temperature and a relative humidity (RH) of 90%.

The solution of Comparative Sample 1 was discharged into the NGI. Fractions of the dose were deposited at different stages of the NGI, in accordance with the particle size of the fraction. Each fraction was washed from the stage and analyzed using HPLC.

The result is shown in Table 8.

TABLE 8
Aerodynamic Particle Size Distribution of Budesonide
Suspension Sample (Comparative Sample 1 (Pulmicort))

| | BD | | |
|---|---|---|---|
| | Dosage | Percentage content | Cut-off diameters |
| Deposited | (μg) | at all levels % | at 30 L/min (μm) |
| Device | 892.42 | 91.86 | |
| Throat | 8.38 | 0.86 | |
| Si | 6.98 | 0.72 | 11.72 |
| S2 | 16.71 | 1.72 | 6.40 |
| S3 | 22.43 | 2.31 | 3.99 |
| S4 | 17.25 | 1.78 | 2.30 |
| S5 | 5.15 | 0.53 | 1.36 |
| S6 | 2.15 | 0.22 | 0.83 |
| S7 | 0 | 0.00 | 0.54 |
| MOC | 0 | 0.00 | |

| | |
|---|---|
| Theoretical dose (μg) | 1000 |
| Actual test dose (μg) | 971.474 |
| Recovery rate (%) | 97.15 |
| ISM (μg) | 63.69 |
| FPD (μg) | 46.98 |
| FPF (%) | 4.84 |

By comparing the NGI parameters of the budesonide suspension of Comparative Sample 1 (Pulmicort) with Sample 2 of the invention, it can be seen that the effective lung deposition of Sample 2 is much higher than that of Comparative Sample 1 (Pulmicort), indicating that the bioavailability of Sample 2 sprayed with the E-flow device is higher.

Because the ISM of Sample 2 is much higher than that of the Comparative Sample 1 (Pulmicort), in order to be consistent with the dose of Pulmicort, it is considered that the effective dose of OH, BD, and TB from Sample 2 can be reduced. According to the invention, the dose of OH is 5.56 μg, the dose of BD is 161.25 μg, and the dose of TB is 6.12 μg. A lower dose can reduce the side effects of a drug on the human body.

### Comparative Example 2

Aerodynamic Particle Size Distribution of budesonide.

Comparative Sample 2: budesonide formulation purchased from AstraZeneca Pty Ltd.

The budesonide suspension sample (Comparative Sample 2) was purchased from AstraZeneca Pty Ltd. Dosage: 160 ug/press, 120 press/bottle, 2 presses/time, twice/day.

The aerodynamic particle size distribution of Comparative Sample 2 was determined using a Next Generation Pharmaceutical Impactor (NGI). The device used to aerosolize the formulation was an e-flow, purchased from PARI in Germany. The device was held close to the NGI inlet until no aerosol was visible. The flow rate of the NGI was set to 30 L/minute and was operated under ambient temperature and a relative humidity (RH) of 90%.

The solution of Comparative sample 2 was discharged into the NGI. Fractions of the dose were deposited at different stages of the NGI, in accordance with the particle size of the fraction. Each fraction was washed from the stage and analyzed using HPLC.

The result are shown in Table 9.

TABLE 9
Aerodynamic Particle Size Distribution of Comparative Sample 2

| | Dosage | Percentage content | Cut-off diameters |
|---|---|---|---|
| BD Deposited | (μg) | at all levels % | at 30 L/min (μm) |
| Device | 24.79 | 8.09 | |
| Throat | 116.32 | 37.95 | |
| Stage 1 | 4.8 | 1.57 | 11.72 |
| Stage 2 | 17.41 | 5.68 | 6.40 |
| Stage 3 | 64.66 | 21.09 | 3.99 |
| Stage 4 | 65.59 | 21.40 | 2.30 |
| Stage 5 | 12.96 | 4.23 | 1.36 |
| Stage 6 | 0 | 0 | 0.83 |
| Stage 7 | 0 | 0 | 0.54 |
| MOC | 0 | 0 | |

| | |
|---|---|
| Theoretical dose (μg) | 320 |
| Actual test dose (μg) | 306.53 |
| Recovery rate (%) | 95.79 |
| ISM | 160.62 μg |
| FPD | 143.21 μg |
| FPF | 46.72% |

The effective lung deposition of the Sample 2 is much higher than that of the Comparative Sample 2.

1. A liquid, propellant-free pharmaceutical formulation comprising:
(a) budesonide, olodaterol, and tiotropium bromide;
(b) a solvent;
(c) a pharmacologically acceptable solubilizing agent.
wherein the pharmaceutical formulation has a pH ranging from about 2.0 to about 6.0.

2. The pharmaceutical formulation according to claim 1, wherein budesonide is present in an amount ranging from about 1 mcg/ml to about 640 mcg/ml; the olodaterol is present in an amount ranging from about 2 mcg/ml to about 500 mcg/ml; and the tiotropium bromide is present in an amount ranging from about 1 mcg/ml to about 200 mcg/ml.

3. The pharmaceutical formulation according to claim 1, wherein the solvent is water substantially free of other solvents.

4. The pharmaceutical formulation according to claim 1, wherein the solubilizing agent is selected from the group consisting of tween-80, cyclodextrin derivatives or a salt thereof, and combinations thereof.

5. The pharmaceutical formulation according to claim 4, wherein the solubilizing agent is present in an amount ranging from about 1 g/100 ml to about 40 g/100 ml.

6. The pharmaceutical formulation according to claim 5, wherein the solubilizing agent is sulfobutylether β-cyclodextrin in an amount ranging from about 0.04 g/4 ml to about 1.6 g/4 ml.

7. The pharmaceutical formulation according to claim 1, further comprising a pharmacologically acceptable preservative selected from the group consisting of benzalkonium chloride, benzoic acid, and sodium benzoate.

8. The pharmaceutical formulation according to claim 8, wherein the pharmacologically acceptable preservative is present in an amount ranging from about 0.08 mg/4 ml to about 12 mg/4 ml.

9. The pharmaceutical formulation according to claim 7, wherein the pharmacologically acceptable preservative is benzalkonium chloride in an amount of about 0.4 mg/4 ml.

10. The pharmaceutical formulation according to claim 1, further comprising a stabilizer selected from the group consisting of edetic acid (EDTA), edetate disodium, edetate disodium dihydrate, and citric acid.

11. The pharmaceutical formulation according to claim 10, wherein the stabilizer is present in an amount ranging from about 0.04 mg/4 ml to about 20 mg/4 ml.

12. The pharmaceutical formulation according to claim 1, further comprising sodium chloride in an amount ranging from about 0.1 g/100 ml to about 0.9 g/100 ml.

13. A method for administering the pharmaceutical formulation according to claim 1, comprising nebulizing a defined amount of the pharmaceutical formulation with an inhaler by using pressure to force the pharmaceutical formulation through a nozzle to form an inhalable aerosol.

14. The method according to claim 13, wherein the defined amount of the pharmaceutical formulation is less than about 8 milliliters of the pharmaceutical formulation.

15. The method according to claim 13, wherein the average particle size of the aerosol is less than about 15 micron.

16. A method of treating asthma or COPD in a patient, comprising administering to the patient the pharmaceutical formulation according to claim 1.

17. The method of claim 16, wherein the pharmaceutical formulation is administered at a therapeutically effective dose of budesonide ranging from about 1 μg to about 100 μg; a therapeutically effective dose of olodaterol ranging from about 5 μg to about 500 μg; and a therapeutically effective dose of tiotropium bromide ranging from about 1 μg to about 100 μg.

18. The liquid, propellant-free pharmaceutical formulation of claim 1 comprising:
an aqueous solution comprising
(a) budesonide in an amount of about 2 mg/100 g;
(b) olodaterol in an amount of about 1.8 mg/100 g
(c) tiotropium bromide in an amount of about 50.9 mg/100 g; and
(d) sulfobutylether β-cyclodextrin in an amount of about 9.6 mg/100 g,
wherein the pH is adjusted with citric acid to a value of 4.0.

19. The liquid, propellant-free pharmaceutical formulation of claim 1 comprising:
an aqueous solution comprising
(a) budesonide in an amount of about 2 mg/100 g;
(b) olodaterol in an amount of about 1.8 mg/100 g
(c) tiotropium bromide in an amount of about 50.9 mg/100 g; and
(d) sulfobutylether β-cyclodextrin in an amount of about 9.6 mg/100 g,
wherein the pH is adjusted with hydrochloric acid to a value of 4.5.

20. The liquid, propellant-free pharmaceutical formulation of claim 1 comprising:
an aqueous solution comprising
(a) budesonide in an amount of about 1 mcg/ml to about 640 mcg/ml;
(b) olodaterol in an amount of about 2 mcg/ml to about 500 mcg/ml;
(c) tiotropium bromide in an amount of about 1 mcg/ml to about 200 mcg/ml; and
(d) sulfobutylether β-cyclodextrin in an amount of about 1 g/100 ml to about 40 g/100 ml,
wherein the pH is adjusted with citric acid to a value of 4.0.

21. The liquid, propellant-free pharmaceutical formulation of claim 1 comprising:
an aqueous solution comprising;
(a) budesonide in an amount of about 1 mcg/ml to about 640 mcg/ml;
(b) olodaterol in an amount of about 2 mcg/ml to about 500 mcg/ml;
(c) tiotropium bromide in an amount of about 1 mcg/ml to about 200 mcg/ml; and
(d) sulfobutylether β-cyclodextrin in an amount of about 1 g/100 ml to about 40 g/100 ml,
wherein the pH is adjusted with hydrochloric acid to a value of 4.5.
